(19) United States
(12) Patent Application Publication
(10) Pub. No.: US 2017/0146539 A1
(43) Pub. Date: May 25, 2017

(54) B7-H3 IN CANCER

(71) Applicant: Mayo Foundation for Medical Education and Research, Rochester, MN (US)

(72) Inventors: Eugene D. Kwon, Rochester, MN (US); Christine M. Lohse, Rochester, MN (US); Yuri Sheinin, Omaha, NE (US); Timothy J. Roth, Elgin, IL (US); Susan M. Harrington, Rochester, MN (US)

(73) Assignee: Mayo Foundation for Medical Education and Research, Rochester, MN (US)

(21) Appl. No.: 15/425,733

(22) Filed: Feb. 6, 2017

Related U.S. Application Data
(63) Continuation of application No. 13/733,724, filed on Jan. 3, 2013.
Application No. 13/733,724 is a division of application No. 12/527,303, filed on Apr. 29, 2010, now abandoned.
(60) Provisional application No. 60/901,558, filed on Feb. 14, 2007.

(51) Int. Cl. G01N 33/574; C07K 16/28; G01N 33/58

(52) U.S. Cl. CPC G01N 33/57434; G01N 33/57438; G01N 33/581; C07K 16/2827; G01N 2333/70532; G01N 2333/908

(57) ABSTRACT

Methods of determining the prognosis of a subject with cancer and determining risk of cancer progression by assessing expression of B7-H3. Methods of reducing B7-H3 levels and/or activity.

### CROSS-REFERENCE TO RELATED APPLICATIONS

This application is a continuation of U.S. application Ser. No. 13/733,724, filed Jan. 3, 2013, which is a divisional of U.S. application Ser. No. 12/527,303 (Abandoned), filed on Apr. 29, 2010, which is a National Stage application under 35 U.S.C. §371 of International Application No. PCT/US2008/053723, having an International Filing Date of Feb. 12, 2008, which claims benefit of priority from U.S. Provisional Application Ser. No. 60/901,558, filed on Feb. 14, 2007. Each of the above applications is incorporated herein by reference in its entirety.

### TECHNICAL FIELD

This document relates using expression levels of B7-H3 to determine the risk of cancer progression or cancer-related death in a subject with cancer.

### BACKGROUND

The incidence of renal cell carcinoma (RCC) has increased steadily over the last three decades, with mortality rates continuing to rise. Jemal et al. (2005) CA Cancer 1 Clin. 55:10-30. To date, the only acceptable treatment for clinically localized RCC is surgical extirpation. Improvements in imaging technology have led to a stage migration and with accompanying surgical advancements, improvements in patient survival have been noted. Pantuck et al. (2001) J. Urol. 166:1611-1623. The five-year survival of RCC patients, however, is still unacceptably low. This low survival rate reflects the 30% of patients who present with metastatic disease, and another 25-30% of patients who subsequently develop disseminated disease after surgical excision of the primary tumor. Motzer et al. (1996) N. Engl. 1 Med. 335:865-875; and Leibovich et al. (2003) Cancer 97:1663-1671. Other treatment modalities for advanced disease such as chemotherapy and radiation have not been shown to be effective. Immunotherapy is the only adjunct therapy available, but less than 10% of patients benefit with durable responses. Fyfe et al. (1995) J. Clin. Oncol. 13:688-696. Limited therapeutic options have done little to improve the median survival of 6-10 months seen in metastatic disease. Figlin et al. (1997) J. Urol. 158:740-750.

Adenocarcinoma of the prostate is the most common non-skin malignancy in elderly men. It is rare before the age of 50, but autopsy studies have found prostatic adenocarcinoma in over half of men more than 80 years old. Although many of these carcinomas are small and clinically insignificant, prostatic adenocarcinoma is second only to lung carcinoma as a cause for tumor-related deaths among males. Prostate adenocarcinomas typically are graded according to the Gleason grading system based on the pattern of growth. There are 5 grades (from 1 to 5) based upon the architectural patterns. Adenocarcinomas of the prostate are given two grades based on the most common and second most common architectural patterns. These two grades are added to get a final Gleason score of 2 to 10. The stage is determined by the size and location of the cancer, whether it has invaded the prostatic capsule or seminal vesicle, and whether it has metastasized. The grade and the stage correlate well with each other and with the prognosis. The prognosis of prostate adenocarcinoma varies widely with tumor stage and grade. Cancers with a Gleason score <6 are generally low grade and not aggressive. Advanced prostate adenocarcinomas typically cause urinary obstruction, and metastasize to regional (pelvic) lymph nodes and to the bones, causing blastic metastases in most cases. Metastases to the lungs and liver also are seen.

Since a large percentage of patients with clinically localized cancers such as RCC and prostate adenocarcinoma subsequently develop metastasis, there is a need for prognostic biomarkers.

### SUMMARY

The present application is based in part on the discovery that B7-H3 expression levels in tumors and in tumor vasculature can be used as indicators of prognosis and risk of cancer progression. For example, increased B7-H3 levels in renal tumors and/or in renal tumor vasculature can be used as a prognostic biomarker for clear cell RCC, while increased B7-H3 levels in prostate tumors can be used as a prognostic biomarker for prostate adenocarcinoma. As described herein, individuals who have clear cell RCC tumors that are positive for B7-H3 (i.e., in which ≧5% of the cells express B7-H3) are at an increased risk of cancer-related death as compared to individuals having clear cell RCC tumors that are negative for B7-H3 (i.e., in which <5% of the cells express B7-H3). In addition, tumor vasculature expression of B7-H3 in subjects with clear cell RCC may provide a target for therapy. Further, individuals who have prostate tumors with cells that stain darkly for B7-H3 (i.e., that have moderate or marked expression of B7-H3) are at an increased risk of prostate cancer progression as compared to individuals having prostate tumors with cells that stain lightly for B7-H3 (i.e., that have weak expression of B7-H3).

In one aspect, this document features a method for assessing the prognosis of a subject with cancer, the method including: (a) assessing in a tissue sample from the subject the level of B7-H3 expression; and (b) if the tissue sample exhibits increased B7-H3 expression relative to the level of B7-H3 expression in a control tissue sample, classifying the subject, in the absence of treatment, as being more likely to die of the cancer as compared to an untreated subject having a corresponding tissue sample that does not exhibit increased B7-H3 expression relative to the level of B7-H3 expression in the control tissue sample, or, if the tissue sample does not exhibit increased B7-H3 expression relative to the level of B7-H3 expression in the control sample, classifying the subject, in the absence of treatment as being less likely to die of the cancer as compared to an untreated subject having a corresponding tissue sample that exhibits increased B7-H3 expression relative to the level of B7-H3 expression in the control tissue sample. Assessing the level of B7-H3 expression can include evaluating the level of polypeptide expression. The evaluating can include fluorescence flow cytometry (FFC), immunohistochemistry, or contacting the tissue sample with an antibody that binds to B7-H3 (e.g., a fluorescently labeled antibody). The tissue sample can be selected from the group consisting of renal, lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. The tissue sample can contain tumor cells. The subject can be a human. The control tissue sample can include tissue from the subject known not be cancerous, or can include corresponding tissue from a subject known not to have the cancer.

In another aspect, this document features a method for determining the prognosis of a subject with cancer, the method including: (a) assessing in a tissue sample from the subject the level of B7-H3 expression; and (b) if the tissue sample exhibits moderate or marked B7-H3 expression, classifying the subject, in the absence of treatment, as being more likely to die of the cancer as compared to an untreated subject having a corresponding tissue sample that exhibits weak or no B7-H3 expression, or, if the tissue sample exhibits weak or no B7-H3 expression, classifying the subject, in the absence of treatment, as being less likely to die of the cancer as compared to an untreated subject having a corresponding tissue sample that exhibits moderate or marked B7-H3 expression. Assessing the level of B7-H3 expression can include evaluating the level of polypeptide expression. The evaluating can include FFC, immunohistochemistry, or contacting the tissue sample with an antibody that binds to B7-H3 (e.g., a fluorescently labeled antibody). The tissue sample can be selected from the group consisting of renal, lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. The tissue sample can contain tumor cells. The cancer can be prostatic adenocarcinoma. The subject can be a human.

In another aspect, this document features a method for determining the risk of cancer progression in a subject with cancer, the method including: (a) assessing in a tissue sample from the subject the level of B7-H3 expression; and (b) if the tissue sample exhibits moderate or marked B7-H3 expression, classifying the subject, in the absence of treatment, as having a greater risk of cancer progression as compared to an untreated subject having a corresponding tissue sample that exhibits weak or no B7-H3 expression, or, if the tissue sample exhibits weak or no B7-H3 expression, classifying the subject, in the absence of treatment, as having a lower risk of cancer progression as compared to an untreated subject having a corresponding tissue sample that exhibits moderate or marked B7-H3 expression. Assessing the level of B7-H3 expression can include evaluating the level of polypeptide expression. The evaluating can include FFC, immunohistochemistry, or contacting the tissue sample with an antibody that binds to B7-H3 (e.g., a fluorescently labeled antibody). The tissue sample can be selected from the group consisting of renal, lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. The tissue sample can contain tumor cells. The cancer can be prostate adenocarcinoma. The subject can be a human.

In another aspect, this document features a method for determining the prognosis of a subject with cancer, the method including: (a) assessing in a tissue sample from the subject the presence or absence of B7-H3 expression; and (b) if the tissue sample is positive for B7-H3 expression, classifying the subject, in the absence of treatment, as being more likely to die of the cancer as compared to an untreated subject having a corresponding tissue sample that is negative for B7-H3 expression, or, if the tissue sample is negative for B7-H3 expression, classifying the subject, in the absence of treatment, as being less likely to die of the cancer as compared to an untreated subject having a corresponding tissue sample that is positive for B7-H3 expression. B7-H3 expression can be assessed by detecting the presence or absence of a B7-H3 polypeptide. The detecting can include FFC, immunohistochemistry, or contacting the tissue sample with an antibody that binds to B7-H3 (e.g., a fluorescently labeled antibody). The tissue sample can be selected from the group consisting of renal, lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. The tissue sample can contain tumor cells. The cancer can be a renal cell carcinoma. The subject can be a human.

In another aspect, this document features a method for determining the prognosis of a subject with cancer, the method including: (a) assessing the presence or absence of B7-H3 expression in the vasculature of a tissue sample from the subject; and (b) if the tissue sample exhibits moderate or diffuse expression of B7-H3 in the vasculature, classifying the subject, in the absence of treatment, as being more likely to die of the cancer as compared to an untreated subject having a corresponding tissue sample that exhibits focal or no B7-H3 expression in the vasculature, or, if the tissue sample exhibits focal or no expression of B7-H3 in the vasculature, classifying the subject, in the absence of treatment, as being less likely to die of the cancer as compared to an untreated subject having a corresponding tissue sample that exhibits moderate or diffuse B7-H3 expression in the vasculature. B7-H3 expression can be assessed by detecting the presence or absence of a B7-H3 polypeptide. The detecting can include FFC, immunohistochemistry, or contacting the tissue sample with an antibody that binds to B7-H3 (e.g., a fluorescently labeled antibody). The tissue sample can be selected from the group consisting of renal, lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. The tissue sample can contain tumor cells. The cancer can be a renal cell carcinoma. The subject can be a human.

In still another aspect, this document features a method of determining risk of cancer progression in a subject with cancer, the method including: (a) assessing in a tissue sample from the subject the presence or absence of B7-H3 expression; and (b) if the tissue sample is positive for B7-H3 expression, classifying the subject, in the absence of treatment, as having a greater risk of cancer progression as compared to an untreated subject having a corresponding tissue sample that is negative for B7-H3 expression, or, if the tissue sample is negative for B7-H3 expression, classifying the subject, in the absence of treatment, as having a lower risk of cancer progression as compared to an untreated subject having a corresponding tissue sample that is positive for B7-H3 expression. B7-H3 expression can be assessed by detecting the presence or absence of a B7-H3 polypeptide. The detecting can include FFC, immunohistochemistry, or contacting the tissue sample with an antibody that binds to B7-H3 (e.g., a fluorescently labeled antibody). The tissue sample can be selected from the group consisting of renal, lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. The tissue sample can contain tumor cells. The cancer can be a renal cell carcinoma. The subject can be a human.

In yet another aspect, this document features a method of determining risk of cancer progression in a subject with cancer, the method including: (a) assessing the presence or absence of B7-H3 expression in the vasculature of a tissue sample from the subject; and (b) if the vasculature exhibits moderate or diffuse B7-H3 expression, classifying the subject, in the absence of treatment, as having a greater risk of cancer progression as compared to an untreated subject having a corresponding tissue sample that exhibits focal or no B7-H3 expression in the vasculature, or, if the vasculature exhibits focal or no B7-H3 expression, classifying the subject, in the absence of treatment, as having a lower risk of cancer progression as compared to an untreated subject having a corresponding tissue sample that exhibits moderate or diffuse B7-H3 expression in the vasculature. B7-H3 expression can be assessed by detecting the presence or absence of a B7-H3 polypeptide. The detecting can include FFC, immunohistochemistry, or contacting the tissue sample with an antibody that binds to B7-H3 (e.g., a fluorescently labeled antibody). The tissue sample can be selected from the group consisting of renal, lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. The tissue sample can contain tumor cells. The cancer can be a renal cell carcinoma. The subject can be a human.

This document also features an article of manufacture comprising an antibody that binds to a B7-H3 polypeptide. The antibody can be labeled (e.g., with a fluorescent label).

In addition, this document features a method for treating prostate cancer in a subject identified as having a tumor in which B7-H3 is expressed, said method comprising administering to said subject an agent that reduces B7-H3 activity. The subject can have a tumor in which B7-H3 is expressed in the tumor cells or in the tumor vasculature. The agent can be a small molecule, an antibody or an antibody fragment, an antisense oligonucleotide, or an interfering RNA.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention will be apparent from the following description, from the drawings and from the claims.

### DESCRIPTION OF DRAWINGS

FIG. 1 is a series of pictures showing variable B7-H3 expression levels in prostate cancer tumors, based on differential staining intensities. FIG. 1A shows weak intensity, FIG. 1B shows moderate intensity, and FIG. 1C shows marked intensity. FIG. 1D demonstrates marked intensity in the setting of perineural invasion (arrowheads).

FIG. 2 is a picture showing marked B7-H3 staining in areas of PIN (arrows), a premalignant lesion associated with prostate cancer. Weak B7-H3 expression in benign prostate epithelium (arrowheads) also is shown.

FIG. 3 is a picture showing B7-H3 expression in normal prostate epithelium (arrowheads) and in malignant tissue (arrows).

FIG. 4 is a graph depicting cancer progression-free survival following radical prostatectomy for prostate cancer patients whose tumors had weak, moderate, and marked B7-H3 intensity, as indicated.

FIG. 5 is a graph depicting cancer-specific survival for clear cell RCC patients with negative tumor B7-H3 expression vs. positive tumor B7-H3 expression.

FIG. 6 is a graph depicting cancer-specific survival for clear cell RCC patients with absent/focal, moderate, and diffuse tumor vasculature B7-H3 expression, as indicated.

FIG. 7 is a graph plotting postoperative prostate-specific antigen (PSA) progression (defined as a postoperative PSA≧0.4 ng/ml) by staining intensity for patients who received neoadjuvant hormonal therapy (NHT).

FIG. 8 is a graph plotting postoperative PSA progression by staining intensity for patients without NHT.

### DETAILED DESCRIPTION

B7-H3 is a member of the B7 family of costimulatory molecules. While the precise function of B7-H3 is unknown, both stimulatory and inhibitory effects on T cell immunity have been identified. As described herein, increased levels of B7-H3 in cancers such as RCC and prostate adenocarcinoma are associated with adverse clinical and pathological aspects of disease, as well as increased risk of cancer progression and cancer-related death. Thus, B7-H3 represents a useful prognostic marker of disease. Moreover, the association of B7-H3 with tumor vasculature in RCC patients makes it a potential molecule to target tumor angiogenesis.

In general, the present document provides methods and materials for determining the prognosis of patients with cancer based on the degree of tumor or vasculature B7-H3 expression (e.g., the number of B7-H3-positive tumor cells or tumor vasculature cells, or the intensity of tumor B7-H3 staining). As used herein, the term “B7-H3” refers to B7-H3 from any mammalian species and the term “hB7-H3” refers to human B7-H3. Further details on B7-H3 polypeptides and nucleic acids are provided in U.S. Pat. No. 6,891,030 and U.S. Publication No. 2005/0202536, the disclosures of which are incorporated herein by reference in their entirety. The amino acid sequence of hB7-H3 can be found in SwissProt under Accession No. Q5ZPR3, and the nucleotide sequence of hB7-H3 can be found in GenBank under Accession No. NM_025240.

### Methods of Determining Risk of Cancer Progression and Cancer-Related Death

Expression of B7-H3 can be used to determine the risk of cancer progression or cancer-related death for a subject with cancer. In general, the methods provided herein include assessing B7-H3 expression in a tissue sample from a subject (e.g., a cancer patient), and correlating increased levels of B7-H3 with an increased risk of cancer progression or increased risk of cancer-related death. In some embodiments, B7-H3 expression levels can be determined based on staining intensity within cells. In some embodiments, B7-H3 expression levels can be determined based on the number of cells that are positive for B7-H3. In some embodiments, B7-H3 expression can be assessed in the vasculature of a tissue sample (e.g., a tumor sample) from a subject. Suitable subjects can be mammals, including, for example, humans, non-human primates such as monkeys, baboons, or chimpanzees, horses, cows (or oxen or bulls), pigs, sheep, goats, cats, rabbits, guinea pigs, hamsters, rats, gerbils, and mice. A “tissue sample” is a sample that contains cells or cellular material. Typically, the tissue sample is from a tumor, e.g., a resection or biopsy of a tumor.

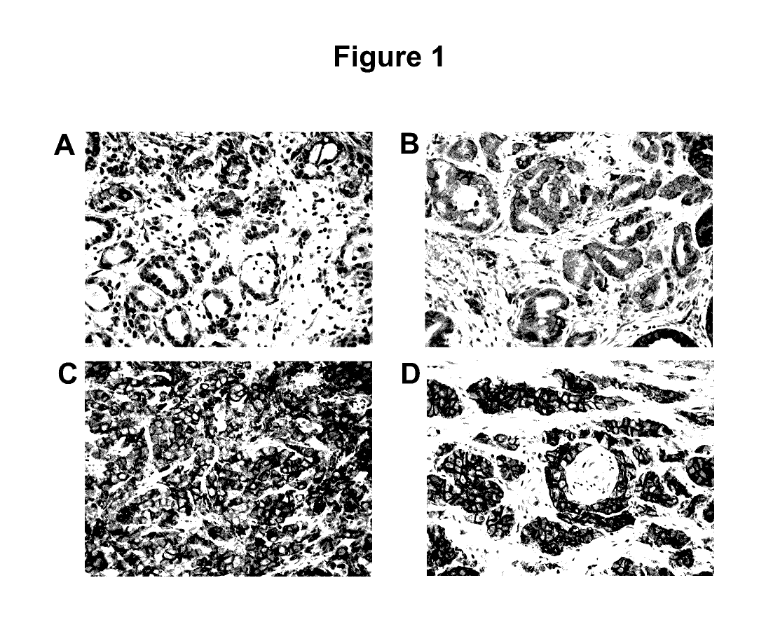

As described herein, subjects having tumors with increased levels of B7-H3 expression can be considered to have a worse prognosis than subjects having tumors that do not demonstrate increased levels of B7-H3 expression. For example, subjects containing B7-H3-positive RCC tumors (i.e., tumors in which ≧5% of the cells express B7-H3) are considered more likely to die from RCC than patients having B7-H3-negative tumors (i.e., tumor in which <5% of the cells express B7-H3). In particular, with respect to RCC, patients with B7-H3-positive tumors are four times more likely to die from RCC than patients with B7-H3-negative tumors. Further, subjects containing prostate tumors in which B7-H3 displays marked expression (e.g., moderate or marked staining intensity, visually assessed as an approximation of the density of the staining; see, e.g., FIGS. 1B and 1C) have an increased risk of cancer progression as compared to subjects containing prostate tumors in which B7-H3 displays weak staining intensity (see, e.g., FIG. 1A). As such, the risk of cancer progression and cancer-related death can be determined, at least in part, by assessing levels of B7-H3. Other factors that can be considered include, for example, the overall health of the patient and previous responses to therapy. Further, assessing expression of B7-H3 can provide valuable clues as to the course of action to be undertaken in treatment of the cancer, as high levels of B7-H3 can indicate a particularly aggressive course of cancer.

In addition, as described in the Examples below, the vast majority of clear cell RCC tumors (98.2% of those examined) appeared to express B7-H3 within the tumor vasculature. Tumor vasculature B7-H3 expression can be characterized as focal, moderate, or diffuse. In particular, tumor vasculature B7-H3 is considered focal if only 5-10% of the tumor vasculature cells are positive for B7-H3, moderate if 10-50% of the tumor vasculature cells are positive for B7-H3, and diffuse if >50% of the tumor vasculature cells are positive for B7-H3. In contrast, only 7.0% of “normal” (i.e., non-tumor) tissue examined had vasculature B7-H3 levels, and all of it was focal. As such, analyzing vasculature expression of B7-H3 can be useful for evaluating a subject (e.g., a human patient), and may provide valuable clues as to the course of action to be undertaken in treatment of the cancer.

Since a number of cancers express B7-H3, the methods provided herein are applicable to a variety of cancers, including, for example, renal cancer, hematological cancer (e.g., leukemia or lymphoma), neurological cancer, melanoma, breast cancer, lung cancer, head and neck cancer, gastrointestinal cancer, liver cancer, pancreatic cancer, genitourinary cancer, bone cancer, and vascular cancer. As such, suitable tissue samples for assessing B7-H3 expression can include, for example, lung, epithelial, connective, vascular, muscle, nervous, skeletal, lymphatic, prostate, cervical, breast, spleen, gastric, intestinal, oral, esophageal, dermal, liver, bladder, renal, thyroid, thymic, adrenal, brain, gallbladder, pancreatic, uterine, ovarian, and testicular tissue. For example, renal, breast, ovarian, and lung tissue samples are particularly useful for determining the prognosis of a patient with RCC, breast, ovarian, or lung cancer, respectively.

In some embodiments, expression of B7-H3 can be tested in leukocytes present in any of the above-listed tissues. Leukocytes infiltrating the tissue can be T lymphocytes (CD4⁺ T cells and/or CD8⁺ T cells) or B lymphocytes. Such leukocytes can also be neutrophils, eosinophils, basophils, monocytes, macrophages, histiocytes, or natural killer cells.

Methods of assessing B7-H3 expression can include evaluating B7-H3 nucleic acid (e.g., mRNA) or polypeptide levels, and can be quantitative, semi-quantitative, or qualitative. Thus, in some embodiments, the level of B7-H3 expression can be determined as a discrete value. For example, where quantitative RT-PCR is used, the level of expression of B7-H3 mRNA can be measured as a numerical value by correlating the detection signal derived from the quantitative assay to the detection signal of a known concentration of: (a) B7-H3 nucleic acid sequence (e.g., B7-H3 cDNA or B7-H3 transcript); or (b) a mixture of RNA or DNA that contains a nucleic acid sequence encoding B7-H3. Alternatively, the level of B7-H3 expression can be assessed using any of a variety of semi-quantitative/qualitative systems known in the art (e.g., immunohistochemistry and/or in situ hybridization). Thus, the level of expression of B7-H3 in a cell or tissue sample can be evaluated as, for example, one or more of “very high”, “high”, “average”, “low”, and/or “very low”; or one or more of “++++”, “+++”, “++”, “+”, “+/−”, and/or “−”. In some embodiments, the level of B7-H3 expression in tissue from a subject can be expressed relative to a control level of B7-H3 expression in, for example, (a) a tissue from the subject known not be cancerous (e.g., a contralateral kidney or lung, normal tissue surrounding or adjacent to a tumor, or an uninvolved lymph node); or (b) a corresponding tissue from one or more other subjects known not to have the cancer of interest, or known not to have any cancer. Thus, B7-H3 expression in a tumor or in tumor vasculature can be considered to be “increased” or “elevated” relative to B7-H3 expression in a control tissue if, for example, a greater number of tumor cells than control cells are positive for B7-H3, if a greater number of tumor vasculature cells than control vasculature cells are positive for B7-H3, or if tumor cells stain more intensely than control cells for B7-H3.

Typically, the presence or absence of B7-H3 expression is determined based on protein expression. As used herein, with respect to B7-H3 protein expression, the term “presence” indicates that ≧5% of the cells in the tissue sample have detectable levels of B7-H3 protein and “absence” indicates that <5% of the cells in the tissue sample have detectable levels of B7-H3 protein.

Any suitable method can be used to detect expression of a protein in a tissue sample, including those known in the art. For example, antibodies that bind to an epitope specific for B7-H3 can be used to assess the presence or absence of B7-H3 expression. As used herein, the terms “antibody” or “antibodies” include intact molecules (e.g., polyclonal antibodies, monoclonal antibodies, humanized antibodies, or chimeric antibodies), as well as fragments thereof (e.g., single chain Fv antibody fragments, Fab fragments, and F(ab)₂ fragments), that are capable of binding to an epitopic determinant of B7-H3 (e.g., hB7-H3). The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). Polyclonal antibodies are heterogeneous populations of antibody molecules that are contained in the sera of the immunized animals. Monoclonal antibodies are homogeneous populations of antibodies to a particular epitope of an antigen.

Antibody fragments that can bind to B7-H3 can be generated using any suitable technique, including those known in the art. For example, F(ab′)₂ fragments can be produced by pepsin digestion of the antibody molecule; Fab fragments can be generated by reducing the disulfide bridges of F(ab′)₂ fragments. Alternatively, Fab expression libraries can be constructed. See, for example, Huse et al. (1989) Science, 246:1275. Once produced, antibodies or fragments thereof can be tested for recognition of B7-H3 using standard immunoassay methods, including ELISA techniques, radioimmunoassays, and Western blotting. See, Short Protocols in Molecular Biology, Chapter 11, Green Publishing Associates and John Wiley & Sons, Edited by Ausubel, F. M et al., 1992.

Antibodies having specific binding affinity for B7-H3 can be produced by, for example, standard methods. See, e.g., Dong et al. (2002) Nature Med. 8:793-800. In general, a B7-H3 polypeptide can be recombinantly produced, or can be purified from a biological sample, and used to immunize animals. As used herein, the term “polypeptide” refers to a polypeptide of at least five amino acids in length. To produce a recombinant B7-H3 polypeptide, a nucleic acid sequence encoding the appropriate polypeptide can be ligated into an expression vector and used to transform a bacterial or eukaryotic host cell. Nucleic acid constructs typically include a regulatory sequence operably linked to a B7-H3 nucleic acid sequence. Regulatory sequences do not typically encode a gene product, but instead affect the expression of the nucleic acid sequence. In bacterial systems, a strain of Escherichia coli such as BL-21 can be used. Suitable E. coli vectors include the pGEX series of vectors that produce fusion proteins with glutathione S-transferase (GST). Transformed E. coli are typically grown exponentially, then stimulated with isopropylthiogalactopyranoside (IPTG) prior to harvesting. In general, such fusion proteins are soluble and can be purified easily from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

Mammalian cell lines that stably express a B7-H3 polypeptide can be produced by using expression vectors with the appropriate control elements and a selectable marker. For example, the eukaryotic expression vector pCDNA.3.1+ (Invitrogen, San Diego, Calif.) can be used to express a B7-H3 polypeptide in, for example, COS cells, Chinese hamster ovary (CHO), or HEK293 cells. Following introduction of the expression vector by electroporation, DEAE dextran, or other suitable method, stable cell lines can be selected. Alternatively, B7-H3 can be transcribed and translated in vitro using wheat germ extract or rabbit reticulocyte lysate.

In eukaryotic host cells, a number of viral-based expression systems can be utilized to express a B7-H3 polypeptide. A nucleic acid encoding a B7-H3 polypeptide can be introduced into an SV40, retroviral or vaccinia based viral vector and used to infect host cells. Alternatively, a nucleic acid encoding a B7-H3 polypeptide can be cloned into, for example, a baculoviral vector and then used to transfect insect cells.

Various host animals can be immunized by injection of the B7-H3 polypeptide. Host animals can include rabbits, chickens, mice, guinea pigs and rats. Various adjuvants that can be used to increase the immunological response depend on the host species, and include Freund's adjuvant (complete and incomplete), mineral gels such as aluminum hydroxide, surface-active substances such as lysolecithin, pluronic polyols, polyanions, peptides, oil emulsions, keyhole limpet hemocyanin and dinitrophenol. Monoclonal antibodies can be prepared using a B7-H3 polypeptide and standard hybridoma technology. In particular, monoclonal antibodies can be obtained by any technique that provides for the production of antibody molecules by continuous cell lines in culture such as described by Kohler et al. (1975) Nature, 256:495, the human B-cell hybridoma technique (Kosbor et al. (1983) Immunology Today, 4:72; Cote et al. (1983) Proc. Natl. Acad. Sci USA, 80:2026), and the EBV-hybridoma technique (Cole et al., Monoclonal Antibodies and Cancer Therapy, Alan R. Liss, Inc., pp. 77-96 (1983)). Such antibodies can be of any immunoglobulin class, including IgG, IgM, IgE, IgA, IgD, and any subclass thereof. The hybridoma producing the monoclonal antibodies provided herein can be cultivated in vitro and in vivo.

Immunohistochemistry refers to the process of localizing proteins in cells of a tissue section, which exploits the principle of antibodies binding specifically to antigens in biological tissues. Immunohistochemical staining can be used in diagnostic clinical procedures as well as in basic research to understand the distribution and localization of biomarkers in different parts of a tissue. Antibody-antigen interactions can be visualized by, for example, using an antibody conjugated to an enzyme (e.g., peroxidase) that can catalyze a color-producing reaction, or to a fluorophore such as fluorescein-5-isothiocyante (FITC), rhodamine, or Texas Red. The antibodies used for specific detection can be polyclonal or monoclonal, although monoclonal antibodies generally are considered to exhibit greater specificity. Immunohistochemical detection of antigens in tissue can be achieved directly or indirectly. The direct method of immunohistochemical staining is a one-step staining method, and involves a labeled antibody (e.g., FITC conjugated antiserum) that reacts directly with the antigen of interest. The indirect method of immunohistochemical staining involves an unlabeled primary antibody that reacts with the antigen of interest, and a secondary antibody that reacts with the primary antibody. The secondary antibody typically is labeled with a fluorescent dye or an enzyme. The secondary antibody also must be against the IgG of the animal species in which the primary antibody was raised. Indirect immunohistochemistry methods typically are more sensitive than direct methods, due to signal amplification through several secondary antibody reactions with different antigenic sites on the primary antibody.

In immunological assays, therefore, an antibody having specific binding affinity for B7-H3, or a secondary antibody that binds to an antibody having specific binding affinity for B7-H3, can be labeled, either directly or indirectly. Suitable labels include, without limitation, radionuclides (e.g., ¹²⁵I, ¹³¹I, ³⁵S, ³H, ³²P, ³³P, or ¹⁴C), fluorescent moieties (e.g., fluorescein, FITC, PerCP, rhodamine, or phycoerythrin), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). Antibodies also can be indirectly labeled by conjugation with biotin and then detected with avidin or streptavidin labeled with a molecule as described above. Methods of detecting or quantifying a label depend on the nature of the label, and include those known in the art. Examples of detectors include, without limitation, x-ray film, radioactivity counters, scintillation counters, spectrophotometers, colorimeters, fluorometers, luminometers, and densitometers. Combinations of these approaches (including “multi-layer” assays) familiar to those in the art can be used to enhance the sensitivity of assays.

Immunological assays for detecting B7-H3 can be performed in a variety of known formats, including sandwich assays (e.g., ELISA assays, sandwich Western blotting assays, or sandwich immunomagnetic detection assays), competition assays (competitive RIA), or bridge immunoassays. See, for example, U.S. Pat. Nos. 5,296,347; 4,233,402; 4,098,876; and 4,034,074. Some protein-detecting assays (e.g., ELISA or Western blot) can be applied to lysates of cells, and others (e.g., immunohistological methods or fluorescence flow cytometry) can be applied to histological sections or unlysed cell suspensions.

In other embodiments, the presence or absence of B7-H3 expression can be determined based on mRNA. As used herein, with respect to mRNA expression, the term “presence” indicates that the tumor sample contains a significantly increased level of mRNA relative to (a) a tissue of a subject known not be cancerous (e.g., a contralateral kidney or lung, or an uninvolved lymph node); or (b) a corresponding tissue from one or more other subjects known not to have the cancer of interest, or known not to have any cancer. As used herein, with respect to mRNA expression, the term “absence” indicates that the tumor sample does not contain a significantly increased level of mRNA relative to (a) a tissue of a subject known not be cancerous; or (b) a corresponding tissue from one or more other subjects known not to have the cancer of interest, or not known to have any cancer.

Any suitable methods for detecting an mRNA in a tissue sample can be used, including, for example, methods known in the art. For example, cells can be lysed and an mRNA in the lysates or in RNA purified or semi-purified from the lysates can be detected by any of a variety of methods including, without limitation, hybridization assays using detectably labeled gene-specific DNA or RNA probes (e.g., Northern Blot assays) and quantitative or semi-quantitative RT-PCR methodologies using appropriate gene-specific oligonucleotide primers. Alternatively, quantitative or semi-quantitative in situ hybridization assays can be carried out using, for example, tissue sections or unlysed cell suspensions, and detectably (e.g., fluorescently or enzyme) labeled DNA or RNA probes. Additional methods for quantifying mRNA include RNA protection assay (RPA) and SAGE.

### Methods for Reducing B7-H3 Activity

This document also provides methods for reducing B7-H3 activity. The methods can include, for example, identifying a subject with a tumor in which B7-H3 is expressed (e.g., in the tumor cells or in the tumor vasculature), and delivering to the subject one or more agents that reduce B7-H3 activity. In some embodiments, the subject can be a human patient. Methods for reducing B7-H3 activity can be used for treatment of cancers such as renal cell carcinoma and prostate adenocarcinoma. The term “treatment” refers to complete abolishment of the symptoms or a decrease in the severity of the symptoms of the disease. In some embodiments, an agent can be administered prophylactically in subjects at risk for developing cancer to prevent development, delay onset, or lessen the severity of subsequently developed disease symptoms. In either case, an effective amount of an agent that reduces B7-H3 activity is administered to the subject. An “effective amount” of an agent is an amount of the agent that is capable of producing a medically desirable result in a treated subject without inducing clinically unacceptable toxicity to the host. The methods can be performed alone or in conjunction with other drugs or therapy (e.g., chemotherapy or radiation).

Suitable agents include, for example, a drug, small molecule, an antibody or an antibody fragment, such as a Fab′ fragment, a F(ab′)₂ fragment, or a scFv fragment that binds B7-H3, an antisense oligonucleotide, an interfering RNA (RNAi), or combinations thereof. Methods for producing antibodies and antibody fragments are described above. Chimeric antibodies and humanized antibodies made from non-human (e.g., mouse, rat, gerbil, or hamster) antibodies also are useful. Chimeric and humanized monoclonal antibodies can be produced by recombinant DNA techniques known in the art, for example, using methods described in U.S. Pat. Nos. 4,816,567; 5,482,856; 5,565,332; 6,054,297; and 6,808,901.

Antisense oligonucleotides provided herein are at least 8 nucleotides in length and hybridize to a B7-H3 transcript. For example, a nucleic acid can be about 8, 9, 10-20 (e.g., 11, 12, 13, 14, 15, 16, 17, 18, 19, or 20 nucleotides in length), 15 to 20, 18 to 25, or 20 to 50 nucleotides in length. In some embodiments, antisense molecules greater than 50 nucleotides in length can be used, including the full-length sequence of a B7-H3 mRNA. As used herein, the term “oligonucleotide” refers to an oligomer or polymer of ribonucleic acid (RNA) or deoxyribonucleic acid (DNA) or analogs thereof. Nucleic acid analogs can be modified at the base moiety, sugar moiety, or phosphate backbone to improve, for example, stability, hybridization, or solubility of a nucleic acid. Modifications at the base moiety include substitution of deoxyuridine for deoxythymidine, and 5-methyl-2′-deoxycytidine and 5-bromo-2′-deoxycytidine for deoxycytidine. Other examples of nucleobases that can be substituted for a natural base include 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone (e.g., an aminoethylglycine backbone) and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone. See, for example, U.S. Pat. Nos. 4,469,863; 5,235,033; 5,750,666; and 5,596,086 for methods of preparing oligonucleotides with modified backbones.

Antisense oligonucleotides also can be modified by chemical linkage to one or more moieties or conjugates that enhance the activity, cellular distribution or cellular uptake of the oligonucleotide. Such moieties include but are not limited to lipid moieties (e.g., a cholesterol moiety); cholic acid; a thioether moiety (e.g., hexyl-S-tritylthiol); a thiocholesterol moiety; an aliphatic chain (e.g., dodecandiol or undecyl residues); a phospholipid moiety (e.g., di-hexadecyl-rac-glycerol or triethyl-ammonium 1,2-di-O-hexadecyl-rac-glycero-3-H-phosphonate); a polyamine or a polyethylene glycol chain; adamantane acetic acid; a palmityl moiety; or an octadecylamine or hexylamino-carbonyl-oxycholesterol moiety. The preparation of such oligonucleotide conjugates is disclosed in, for example, U.S. Pat. Nos. 5,218,105 and 5,214,136.

Methods for synthesizing antisense oligonucleotides are known, including solid phase synthesis techniques. Equipment for such synthesis is commercially available from several vendors including, for example, Applied Biosystems (Foster City, Calif.). Alternatively, expression vectors that contain a regulatory element that directs production of an antisense transcript can be used to produce antisense molecules.

Antisense oligonucleotides can bind to a nucleic acid encoding B7-H3, including DNA encoding B7-H3 RNA (including pre-mRNA and mRNA) transcribed from such DNA, and also cDNA derived from such RNA, under physiological conditions (i.e., physiological pH and ionic strength). For example, an antisense oligonucleotide can hybridize under physiological conditions to the nucleotide sequence set forth in GenBank Accession No. AY280972.

It is understood in the art that the sequence of an antisense oligonucleotide need not be 100% complementary to that of its target nucleic acid to be hybridizable under physiological conditions. Antisense oligonucleotides hybridize under physiological conditions when binding of the oligonucleotide to the B7-H3 nucleic acid interferes with the normal function of the B7-H3 nucleic acid, and non-specific binding to non-target sequences is minimal.

Target sites for B7-H3 antisense oligonucleotides include the regions encompassing the translation initiation or termination codon of the open reading frame (ORF) of the gene. In addition, the ORF has been targeted effectively in antisense technology, as have the 5′ and 3′ untranslated regions. Furthermore, antisense oligonucleotides have been successfully directed at intron regions and intron-exon junction regions. Further criteria can be applied to the design of antisense oligonucleotides. Such criteria are well known in the art, and are widely used, for example, in the design of oligonucleotide primers. These criteria include the lack of predicted secondary structure of a potential antisense oligonucleotide, an appropriate G and C nucleotide content (e.g., approximately 50%), and the absence of sequence motifs such as single nucleotide repeats (e.g., GGGG runs). The effectiveness of antisense oligonucleotides at modulating expression of a B7-H3 nucleic acid can be evaluated by measuring levels of the B7-H3 mRNA or polypeptide (e.g., by Northern blotting, RT-PCR, Western blotting, ELISA, or immunohistochemical staining).

Double-stranded interfering RNA (RNAi) homologous to B7-H3 DNA also can be used to reduce expression of B7-H3 and consequently, activity of B7-H3. See, e.g., U.S. Pat. No. 6,933,146; Fire et al. (1998) Nature 391:806-811; Romano and Masino (1992) Mol. Microbiol. 6:3343-3353; Cogoni et al. (1996) EMBO J. 15:3153-3163; Cogoni and Masino (1999) Nature 399:166-169; Misquitta and Paterson (1999) Proc. Natl. Acad. Sci. USA 96:1451-1456; and Kennerdell and Carthew (1998) Cell 95:1017-1026.

The sense and anti-sense RNA strands of RNAi can be individually constructed using chemical synthesis and enzymatic ligation reactions using procedures known in the art. For example, each strand can be chemically synthesized using naturally occurring nucleotides or nucleic acid analogs. The sense or anti-sense strand also can be produced biologically using an expression vector into which a target B7-H3 sequence (full-length or a fragment) has been subcloned in a sense or anti-sense orientation. The sense and anti-sense RNA strands can be annealed in vitro before delivery of the dsRNA to cells. Alternatively, annealing can occur in vivo after the sense and anti-sense strands are sequentially delivered to the tumor vasculature or to tumor cells.

In one embodiment, the agent (e.g., drug, small molecule, antibody, antibody fragment, antisense oligonucleotide, interfering RNA) itself is administered to a subject. Generally, the agent will be suspended in a pharmaceutically-acceptable carrier (e.g., physiological saline) and administered orally or by intravenous infusion, or injected subcutaneously, intramuscularly, intrathecally, intraperitoneally, intrarectally, intravaginally, intranasally, intragastrically, intratracheally, or intrapulmonarily. The agent can, for example, be delivered directly to the affected organ or tissue and/or vasculature of the organ, or a site of an immune response such as a lymph node in the region of an affected tissue or organ or spleen. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.0001-100.0 mg/kg. Wide variations in the needed dosage are to be expected in view of the variety of compounds available and the differing efficiencies of various routes of administration. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Alternatively, a nucleic acid (e.g., an expression vector containing a regulatory sequence operably linked to a nucleic acid encoding the polypeptide, an expression vector containing a regulatory sequence operably linked to a nucleic acid encoding the antisense oligonucleotide, or an expression vector from which sense and anti-sense RNAs can be transcribed under the direction of separate promoters, or a single RNA molecule containing both sense and anti-sense sequences can be transcribed under the direction of a single promoter) can be delivered to appropriate cells in a subject. Suitable expression vectors include plasmids and viral vectors such as herpes viruses, retroviruses, vaccinia viruses, attenuated vaccinia viruses, canary pox viruses, adenoviruses and adeno-associated viruses, among others.

Expression of the nucleic acids can be directed to any cell in the body of the subject. However, it is particularly useful to direct expression to cells in, or close to, lymphoid tissue draining an affected tissue or organ. Expression of the nucleic acid can be directed, for example, to cells comprising the tumor vasculature, cancer tissue (e.g., tumor cells) or immune-related cells, e.g., B cells, macrophages/monocytes, or interdigitating dendritic cells. This can be achieved by, for example, the use of polymeric, biodegradable microparticle or microcapsule delivery devices known in the art and/or tissue or cell-specific antibodies. Alternatively, tissue specific targeting can be achieved by the use of tissue-specific transcriptional regulatory sequences (i.e., tissue specific promoter) which are known in the art.

Nucleic acids can be delivered to cells using liposomes, which can be prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells [Cristiano et al. (1995) J. Mol. Med. 73:479]. Delivery of “naked DNA” (i.e., without a delivery vehicle) to an intramuscular, intradermal, or subcutaneous site is another means to achieve in vivo expression.

Nucleic acids can be administered in a pharmaceutically acceptable carrier. Pharmaceutically acceptable carriers are biologically compatible vehicles that are suitable for administration to a human, e.g., physiological saline or liposomes. As discussed above, the dosage for any one patient depends upon many factors, including the patient's size, body surface area, age, the particular compound to be administered, sex, time and route of administration, general health, and other drugs being administered concurrently. Dosages will vary, but a preferred dosage for administration of nucleic acid is from approximately 10⁶ to approximately 10¹² copies of the nucleic acid. This dose can be repeatedly administered, as needed. Routes of administration can be any of those described above.

In addition, the method can be an ex vivo procedure that involves providing a recombinant cell which is, or is a progeny of a cell, obtained from a subject and has been transfected or transformed ex vivo with one or more nucleic acids encoding one or more agents that reduce B7-H3 activity, so that the cell expresses the agent(s); and administering the cell to the subject. The cells can be cells obtained from a cancer tissue (e.g., tumor cells) or from a non-cancerous tissue obtained preferably from a subject to whom these cells are to be administered or from another subject. The donor and recipient of the cells can have identical major histocompatibility complex (MHC; HLA in humans) haplotypes. Optimally, the donor and recipient are homozygotic twins or are the same individual (i.e., are autologous). The recombinant cells can also be administered to recipients that have no, or only one, two, three, or four MHC molecules in common with the recombinant cells, e.g., in situations where the recipient is severely immunocompromised, where only mismatched cells are available, and/or where only short term survival of the recombinant cells is required or desirable.

The efficacy of an agent can be evaluated both in vitro and in vivo. Briefly, the agent can be tested for its ability, for example, to (a) reduce B7-H3 activity, (b) inhibit growth of cancer cells, (c) induce death of cancer cells, or (d) render the cancer cells more susceptible to cell-mediated immune responses generated by leukocytes (e.g., lymphocytes and/or macrophages). For in vivo studies, the agent can, for example, be injected into an animal (e.g., a mouse cancer model) and its effects on cancer are then assessed. Based on the results, an appropriate dosage range and administration route can be determined.

In some embodiments, one or more supplementary agents can be administered with an agent that reduces B7-H3 activity. Suitable supplementary agents include, for example, immunomodulatory cytokines, growth factors, anti-angiogenic factors, immunogenic stimuli, and/or antibodies specific for any of these. Such supplementary agents can administered before, simultaneously with, or after delivery of an agent that reduces B7-H3 activity.

Examples of immunomodulatory cytokines, growth factors, and anti-angiogenic factors include, without limitation, interleukin (IL)-1 to 25 (e.g., IL-2, IL-12, or IL-15), interferon-γ (IFN-γ), interferon-α (IFN-α), interferon-β (IFN-β), tumor necrosis factor-α (TNF-α), granulocyte macrophage colony stimulating factor (GM-CSF), endostatin, angiostatin, and thrombospondin. Immunomodulatory cytokines, growth factors, anti-angiogenic factors include substances that serve, for example, to inhibit infection (e.g., standard anti-microbial antibiotics), inhibit activation of T cells, or inhibit the consequences of T cell activation. For example, where it is desired to decrease a Th1-type immune response (e.g., in a delayed type hypersensitivity response), a cytokine such as interleukin (IL)-4, IL-10, or IL-13 or an antibody specific for a cytokine such as IL-12 or interferon-γ (IFN-γ) can be used. Alternatively, where it is desired to inhibit a Th2-type immune response (e.g., in an immediate type hypersensitivity response), a cytokine such as IL-12 or IFN-γ or an antibody specific for IL-4, IL-10, or IL-13 can be used as a supplementary agent. Also of interest are antibodies (or any of the above-described antibody fragments or derivatives) specific for proinflammatory cytokines and chemokines such as IL-1, IL-6, IL-8, TNF-α, macrophage inflammatory protein (MIP)-1, MIP-3α, monocyte chemoattractant protein-1 (MCP-1), epithelial neutrophil activating peptide-78 (ENA-78), interferon-γ-inducible protein-10 (IP10), Rantes, and any other appropriate cytokine or chemokine recited herein.

### Articles of Manufacture

One or more antibodies that can bind to a B7-H3 polypeptide (e.g., hB7-H3), or one or more nucleic acids that can bind to a B7-H3 nucleic acid can be combined with packaging material and sold as a kit for detecting B7-H3 from biological samples, determining prognosis of a subject with cancer, or determining risk of cancer progression in a subject. Components and methods for producing articles of manufactures are well known. In addition, the articles of manufacture may further include reagents such as secondary antibodies, sterile water, pharmaceutical carriers, buffers, indicator molecules, solid phases (e.g., beads), and/or other useful reagents (e.g., positive and negative controls) for detecting B7-H3 from biological samples, determining prognosis of a subject with cancer, or determining risk of cancer progression in a subject. The antibodies can be in a container, such as a plastic, polyethylene, polypropylene, ethylene, or propylene vessel that is either a capped tube or a bottle. In some embodiments, the antibodies can be included on a solid phase such as a handheld device for bedside testing. Instructions describing how the various reagents are effective for determining prognosis of a subject with cancer or determining risk of cancer progression also may be included in such kits.

The invention will be further described in the following examples, which do not limit the scope of the invention described in the claims.

### EXAMPLES

### Example 1—Association of B7-H3 Staining Intensity with Prostate Adenocarcinoma

### 1. Materials and Methods

Patient Selection—

Four hundred and fifty-four (454) consecutive patients were identified who had biopsy-proven diagnoses of prostate adenocarcinoma and who were subsequently treated with radical retropubic prostatectomy (RRP), without neoadjuvant hormonal therapy, between 1995 and 1998 (Sebo et al, Cancer 2001; 91:2196-2204). For the purpose of this analysis, 19 patients with positive lymph nodes at RRP were excluded.

Clinical and RRP Pathologic Features—

The clinical and RRP pathologic features evaluated included preoperative serum prostate specific antigen (PSA), tumor volume, Gleason score, seminal vesicle involvement, surgical margins, and extraprostatic extension. Preoperative serum PSA values were expressed as ng/mL. The prostate glands were evaluated at the time of surgery by a standardized, limited sampling protocol using frozen section technique, followed by reevaluation the following day with hematoxylin and eosin-stained permanent sections (DiMarco et al. (2003) Urologic Oncol. 21:439-446). All gross specimens were inked. The prostate specimen, in a fresh state, was initially assessed by examining microscopically five surgical margins (right and left apex, right and left bladder base, and distal urethra). This was followed by at least eight standard sections through the peripheral zone of the right and left sides and right and left seminal vesicles. The average number of tissue sections evaluated per RRP specimen was 14 (range 13-63). The number of sections was contingent upon tumor volume. Specifically, more sections were submitted for microscopic examination for small tumors to get a more accurate estimate of tumor volume. Crude estimates of tumor volume in cubic centimeters (cc) were calculated using the three-dimensional measurements of the tumor at the time of initial evaluation. During sectioning of the fresh prostate gland tissue, microscopic identification of cancer in the frozen sections derived from the prostate were correlated with the gross locations of the sections taken for histologic evaluation. The combination of the gross and microscopic observations served as the framework for the tumor volume estimates. Two urologic pathologists reviewed the RRP specimens, with consensus, for Gleason score, seminal vesicle involvement, surgical margins, and extraprostatic extension. Extraprostatic extension was defined as seminal vesicle involvement or malignant cells outside the prostatic capsule in adipose or ganglion tissue. These clinical and pathologic features were also combined into the GPSM score (Blute et al. (2001) J. Urol. 165:119-125), which uses preoperative serum PSA and RRP Gleason score, seminal vesicle involvement, and positive surgical margins.

Patient Outcome—

Digital rectal exams and serum PSAs were performed every three or four months for the first two years following RRP, every six months for the next three years, and annually thereafter (Sebo et al. (2002) Am. J. Surg. Pathol. 26:431-439). Systemic progression was determined by bone scan or computerized tomography. Local recurrence was determined by clinical examination or needle biopsy. Cancer progression was defined as a postoperative PSA level of 0.4 ng/mL or greater, local recurrence, or systemic progression.

B7-H3 Immunohistochemistry—

Paraffin-embedded tissue sections were cut into 5-micron sections, deparaffinized in xylene, and rehydrated in a graded series of ethanols. Antigen retrieval was performed by heating tissue sections in EDTA 1 mM pH 8 to 121° C. using a Digital Decloaking Chamber (Biocare Medical, Walnut Creek, Calif.), cooling to 90° C., and incubating for an additional 5 minutes before opening the Decloaking Chamber. Sections were washed in running DH₂O for 5 minutes and then incubated for 5 minutes in Wash Buffer (Dako #S3006) before being placed on the Autostainer Plus (Dako) for the following protocol. Sections were blocked for endogenous peroxidase for 5 minutes using Endogenous Blocking Solution (Dako #S2001), washed twice in wash buffer, and incubated 5 minutes in Serum-Free Protein Block (Dako #X0909) followed by incubation for 60 minutes in purified goat anti-human B7-H3 antibody (R&D Systems #AF1027, 100 ug/ml) diluted 1:80 with DaVinci Green antibody diluent (Biocare Medical #PD900M). Sections were washed in wash buffer and incubated for 15 minutes in goat probe from Goat HRP-Polymer Kit (Biocare Medical #GHP516L), washed in wash buffer, and then incubated for 15 minutes with goat polymer from Goat HRP-Polymer Kit. Sections were then washed with wash buffer and visualized by incubating in Betazoid DAB (Biocare Medical #BDB2004L) for 5 minutes. Sections were washed with DH₂O, counterstained with hematoxylin, dehydrated in ethanol, cleared in xylene, and coverslipped with permanent mounting media.

B7-H3 Quantitation—

The percents of tumor and adjacent non-tumor cells that stained positive for B7-H3 were quantified in 10% increments. In addition, the intensity of B7-H3 staining was recorded as absent, weak, moderate, or marked.

Statistical Methods—

Cancer progression following RRP was estimated using the Kaplan-Meier method. The duration of follow-up was calculated from the date of RRP to the date of cancer progression, last follow-up, or the last postoperative serum PSA measurement. Comparisons of cancer progression between patients with and without archived tissue available for study were evaluated using a log-rank test. Tumor and non-tumor B7-H3 expression were compared using the signed rank test. Associations of B7-H3 intensity with clinical and RRP pathologic features were evaluated using Kruskal-Wallis and chi-square tests, while associations of B7-H3 intensity with cancer progression following RRP were evaluated using Cox proportional hazards regression models. Statistical analyses were performed using the SAS software package (SAS Institute; Cary, N.C.). All tests were two-sided and p-values <0.05 were considered statistically significant.

### 2. Results

Comparison of Patients with and without Tissue—

Three hundred and thirty-eight (78%) of the 435 eligible patients who had archived paraffin-embedded tissue were available for study. There was no statistically significant difference in cancer progression following RRP between patients with and without tissue available for study (p=0.289).

Clinical and RRP Pathologic Features and Patient Outcome—

The clinical and pathologic features studied are summarized in Table 1. At last follow-up, 93 of the 338 patients studied experienced cancer progression at a median of 3.9 years following RRP (range 0.1-9.7). Among the 245 patients who did not progress, the median duration of follow-up was 9.1 years (range 0.1-11.3). The estimated cancer progression-free survival rates (standard error [SE], number still at risk) at 1, 3, 5, and 7 years following RRP were 95.0% (1.2%, 319), 88.0% (1.8%, 286), 81.1% (2.2%, 256), and 75.6% (2.4%, 220), respectively.

B7-H3 Expression—

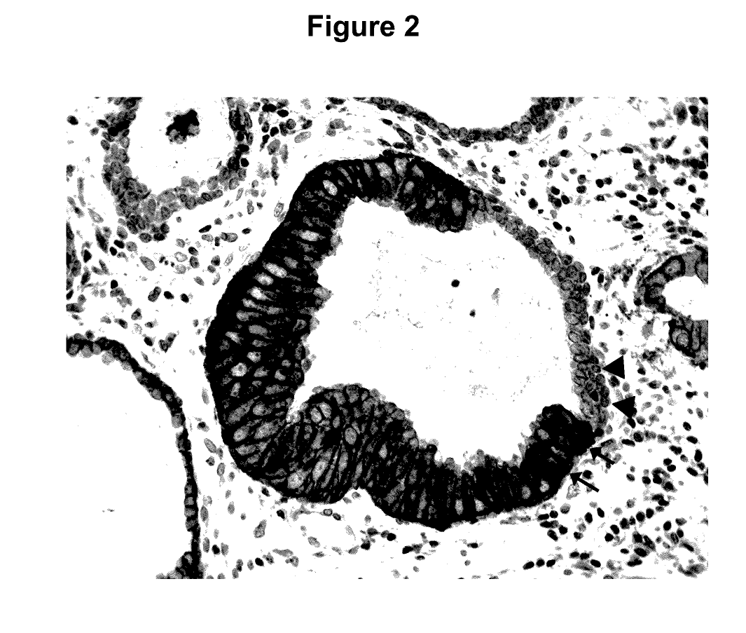

All 338 cases had positive tumor B7-H3 expression, ranging from 40% to 100%. In fact, 282 (83.4%) of the cases had 100% tumor B7-H3 expression. However, the intensity of B7-H3 staining varied. Sixty-five (19.2%) cases had weak tumor B7-H3 intensity (FIG. 1A), 206 (61.0%) had moderate tumor B7-H3 intensity (FIG. 1B), and 67 (19.8%) had marked tumor B7-H3 intensity (FIG. 1C). Marked tumor B7-H3 intensity was seen mostly in large neoplastic glands, with staining that was primarily circumferential cell membranous and cytoplasmic. Marked intensity also was observed in areas of perineural invasion (FIG. 1D, arrowheads). In addition, marked B7-H3 staining was observed in areas of PIN (FIG. 2, arrows), a premalignant lesion associated with prostate cancer. Those cases with fewer than 100% B7-H3-positive tumor cells were generally seen in small neoplastic foci of low Gleason grade.

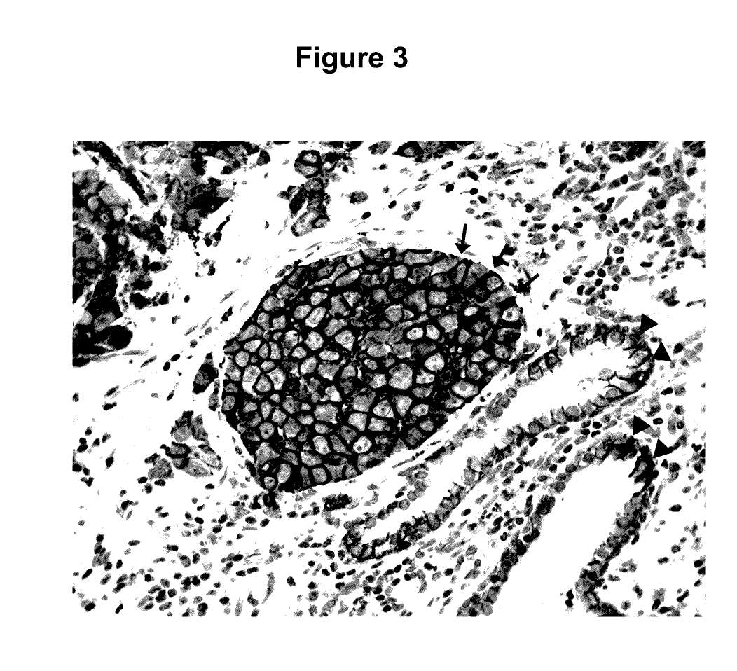

All but two cases had areas of normal, atrophic, or hyperplastic prostatic epithelium, which also demonstrated B7-H3 expression ranging from 20% to 100%. The intensity of non-tumor B7-H3 expression was weak in most cases (224; 66.7%) (FIG. 3, arrowheads), while 111 (33.0%) cases had moderate intensity and only 1 (0.3%) case had marked non-tumor B7-H3 intensity. Tumor B7-H3 expression was significantly higher than non-tumor B7-H3 expression (p<0.001; signed rank test).

Atrophic prostatic ducts and acini showed no or, rarely, weak B7-H3 staining. Hyperplastic glands showed weak to moderate partial membranous staining, with positive basal and lateral surfaces and negative apical surfaces. This distribution corresponded to the apocrine compartment of prostatic epithelium. Areas of prostatic intraepithelial neoplasia tended to have marked membranous and cytoplasmic staining, while seminal vesicles were completely negative. No B7-H3 staining was seen in tumor blood vessels, with the exception of inflammatory areas that contained granulation tissue. In these areas, newly formed small blood vessels demonstrated weak staining.

Association with B7-H3 Expression—

A comparison of tumor B7-H3 intensity with the clinical and RRP pathologic features studied is shown in Table 2. Tumor B7-H3 intensity was statistically significantly associated with larger tumor volume, extraprostatic extension, higher GPSM score, and three of the four components of the GPSM score including higher Gleason score, seminal vesicle involvement, and positive surgical margins. For example, none of the tumors with weak B7-H3 intensity had Gleason scores of 8 or 9, compared with 10 (4.9%) and 23 (34.3%) of the tumors with moderate and marked B7-H3 intensity, respectively (p<0.001).

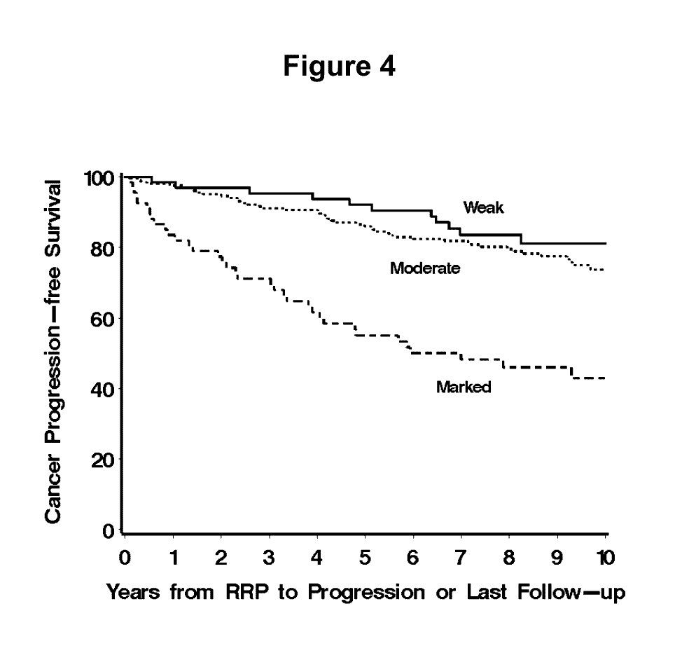

Univariately, patients with tumors of moderate B7-H3 intensity were 35% more likely to experience cancer progression following RRP compared to patients with tumors of weak intensity, but this difference was not statistically significant (risk ratio 1.35; 95% CI 0.70-2.61; p=0.369). On the other hand, patients with tumors of marked B7-H3 intensity were over four times more likely to progress compared to patients with tumors of weak intensity (risk ratio 4.42; 95% CI 2.24-8.72; p<0.001). The estimated cancer progression-free survival rates (SE, number still at risk) at 5 years following RRP were 92.1% (3.4%, 57), 86.0% (2.4%, 166), and 55.0% (6.2%, 33) for patients with tumors of weak, moderate, and marked B7-H3 intensity, respectively (FIG. 4).

The associations of tumor B7-H3 intensity with cancer progression after adjusting for each of the clinical and RRP pathologic features studied are shown in Table 3. Marked tumor B7-H3 intensity was significantly associated with cancer progression, even after multivariate adjustment. For example, after accounting for the association of GPSM score with cancer progression, patients with tumors of marked B7-H3 intensity were still over twice as likely to progress compared to patients with tumors of weak B7-H3 intensity (risk ratio 2.20; 95% CI 1.03-4.70; p=0.042).

TABLE 1
Summary of Clinical and RRP Pathologic Features
for 338 Patients with Prostate Adenocarcinoma

| Feature | Median (Range) |
|---|---|
| Preoperative Serum PSA in | 6.3 (0.6-112.0) |
| ng/mL (N = 324) | |
| Tumor Volume in cc (N = 334) | 2.7 (0.0005-67.5) |
| GPSM Score (N = 324) | 8 (6-16) |
| Gleason Score | N (%) |
| 5 | 6 (1.8) |
| 6 | 158 (46.8) |
| 7 | 141 (41.7) |
| 8 | 18 (5.3) |
| 9 | 15 (4.4) |
| Seminal Vesicle Involvement | |
| Absent | 303 (89.6) |
| Present | 35 (10.4) |
| Surgical Margins | |
| Negative | 197 (58.3) |
| Positive | 141 (41.7) |
| Extraprostatic Extension | |
| Absent | 268 (79.3) |
| Present | 70 (20.7) |

TABLE 2
Comparison of Clinical and RRP Pathologic
Features by Tumor B7-H3 Intensity

| | Tumor B7-H3 Intensity | | | |
|---|---|---|---|---|
| | Weak | Moderate | Marked | |
| | N = 65 | N = 206 | N = 67 | |
| Feature | Median (Range) | | | P-value |
| Preoperative Serum | 5.5 (1.1-22.7) | 6.1 (0.6-39.7) | 7.3 (1.0-112.0) | 0.056 |
| PSA in ng/mL | | | | |
| Tumor Volume in cc | 1.0 (0.0005-26.3) | 2.6 (0.004-67.5) | 6.1 (0.024-60.0) | <0.001 |
| GPSM Score | 7 (6-12) | 8 (6-15) | 10 (6-16) | <0.001 |

N (%)

| | | | | |
|---|---|---|---|---|
| Gleason Score | | | | |
| 5 or 6 | 49 (75.4) | 106 (51.5) | 9 (13.4) | <0.001 |
| 7 | 16 (24.6) | 90 (43.7) | 35 (52.2) | |
| 8 or 9 | 0 (0.0) | 10 (4.9) | 23 (34.3) | |
| Seminal Vesicle | | | | |
| Involvement | | | | |
| Absent | 62 (95.4) | 191 (92.7) | 50 (74.6) | <0.001 |
| Present | 3 (4.6) | 15 (7.3) | 17 (25.4) | |
| Surgical | | | | |
| Margins | | | | |
| Negative | 50 (76.9) | 118 (57.3) | 29 (43.3) | <0.001 |
| Positive | 15 (23.1) | 88 (42.7) | 38 (56.7) | |
| Extraprostatic | | | | |
| Extension | | | | |
| Absent | 61 (93.9) | 171 (83.0) | 36 (53.7) | <0.001 |
| Present | 4 (6.1) | 35 (17.0) | 31 (46.3) | |

TABLE 3
Association of B7-H3 Intensity with
Cancer Progression following RRP

| | | |
|---|---|---|
| | Risk Ratio | |
| Feature | (95% CI) | P-value |

B7-H3 Intensity

| | | |
|---|---|---|
| Weak | 1.0 (reference) | |
| Moderate | 1.35 (0.70-2.61) | 0.369 |
| Marked | 4.42 (2.24-8.72) | <0.001 |
| Preoperative Serum PSA¹ | 1.44 (1.12-1.86) | 0.005 |
| Weak | 1.0 (reference) | |
| Moderate | 1.24 (0.64-2.39) | 0.530 |
| Marked | 3.60 (1.80-7.20) | <0.001 |
| Tumor Volume¹ | 1.26 (1.08-1.45) | 0.002 |
| Weak | 1.0 (reference) | |
| Moderate | 1.11 (0.57-2.15) | 0.757 |
| Marked | 2.92 (1.43-5.94) | 0.003 |
| GPSM Score | 1.23 (1.13-1.35) | <0.001 |
| Weak | 1.0 (reference) | |
| Moderate | 1.07 (0.55-2.08) | 0.848 |
| Marked | 2.20 (1.03-4.70) | 0.042 |
| Gleason | | |
| Score | | |
| 5 or 6 | 1.0 (reference) | |
| 7 | 2.32 (1.39-3.86) | 0.001 |
| 8 or 9 | 3.97 (1.96-8.05) | <0.001 |
| Weak | 1.0 (reference) | |
| Moderate | 1.09 (0.56-2.12) | 0.801 |
| Marked | 2.25 (1.05-4.82) | 0.036 |
| Seminal | | |
| Vesicle | | |
| Involvement | | |
| Absent | 1.0 (reference) | |
| Present | 2.00 (1.17-3.42) | 0.011 |
| Weak | 1.0 (reference) | |
| Moderate | 1.33 (0.69-2.57) | 0.390 |
| Marked | 3.64 (1.81-7.35) | <0.001 |
| Surgical | | |
| Margins | | |
| Negative | 1.0 (reference) | |
| Positive | 1.70 (1.12-2.60) | 0.013 |
| Weak | 1.0 (reference) | |
| Moderate | 1.21 (0.62-2.35) | 0.571 |
| Marked | 3.66 (1.83-7.33) | <0.001 |
| Extraprostatic | | |
| Extension | | |
| Absent | 1.0 (reference) | |
| Present | 2.14 (1.36-3.36) | 0.001 |
| Weak | 1.0 (reference) | |
| Moderate | 1.23 (0.64-2.39) | 0.532 |
| Marked | 3.21 (1.57-6.55) | 0.001 |

¹Analyzed on the natural log scale. As such, the risk ratio represents a 1-unit increase in the feature listed on the natural log scale, not the original scale.

### Example 2—Tumor and Tumor Vasculature B7-H3 Expression in Renal Cell Carcinoma

Permanently fixed specimens from 327 patients who underwent nephrectomy for unilateral, sporadic, non-cystic clear cell RCC between 1990 and 1994 were immunohistochemically stained for B7-H3. At last follow-up, 201 of the 327 patients under study had died, including 110 who died from RCC at a median of 2.4 years following surgery (range 0.2-14.1). Among the 126 surviving patients, the median duration of follow-up was 12.6 years (range 0.1-16.7). All but 5 of the patients still alive at last follow-up had at least 10 years of follow-up.

The association of B7-H3 expression with outcome was evaluated with Cox proportional hazards regression models. Positive tumor expression of B7-H3 (defined as ≧5% tumor cells positive for B7-H3) was identified in 58 (17.7%) patients. Positive tumor B7-H3 expression was significantly associated with symptoms at presentation, presence and level of tumor thrombus, larger tumor size, renal sinus fat invasion, higher primary tumor classification, regional lymph node involvement, higher TNM stage group, higher nuclear grade, coagulative tumor necrosis, and sarcomatoid differentiation. Some of these differences were quite dramatic. For example, 53 (91.4%) of the 58 tumors with positive tumor B7-H3 expression were high grade, compared with only 91 (33.8%) of the 269 patients with negative tumor B7-H3 expression (p<0.001).

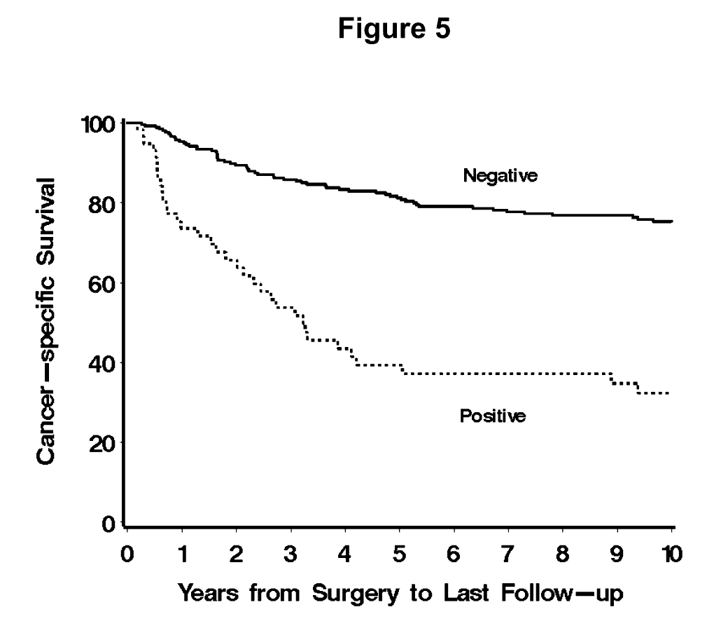

Positive tumor B7-H3 expression increased the risk of death from clear cell RCC nearly 4-fold (risk ratio 3.78; 95% CI 2.52-5.65; p<0.001). The 10-year cancer-specific survival rate for patients with negative tumor B7-H3 expression was 75.3% compared with 32.3% for patients with positive tumor B7-H3 expression (FIG. 5). However, this difference was not statistically significant after adjusting for the SSIGN score (risk ratio 1.22; 95% CI 0.78-1.91; p=0.394).

Only 6 tumors (1.8%) did not have any tumor vasculature B7-H3 expression. There were 81 tumors (24.8%) with focal tumor vasculature B7-H3 expression, 96 (29.4%) with moderate tumor vasculature B7-H3 expression, and 144 (44.0%) with diffuse tumor vasculature B7-H3 expression. The level of tumor vasculature B7-H3 expression was categorized as absent, focal (5-10%), moderate (>10-50%), and diffuse (>50%). Tumor vasculature expression also was significantly associated with a number of clinical and pathologic features, including symptoms at presentation, larger tumor size, fat invasion, higher primary tumor classification, higher TNM stage group, higher nuclear grade, and coagulative tumor necrosis. For example, the proportion of high grade tumors was 28.7%, 37.5%, and 57.6% among tumors with absent/focal, moderate, and diffuse tumor vasculature B7-H3 expression, respectively (p<0.001).

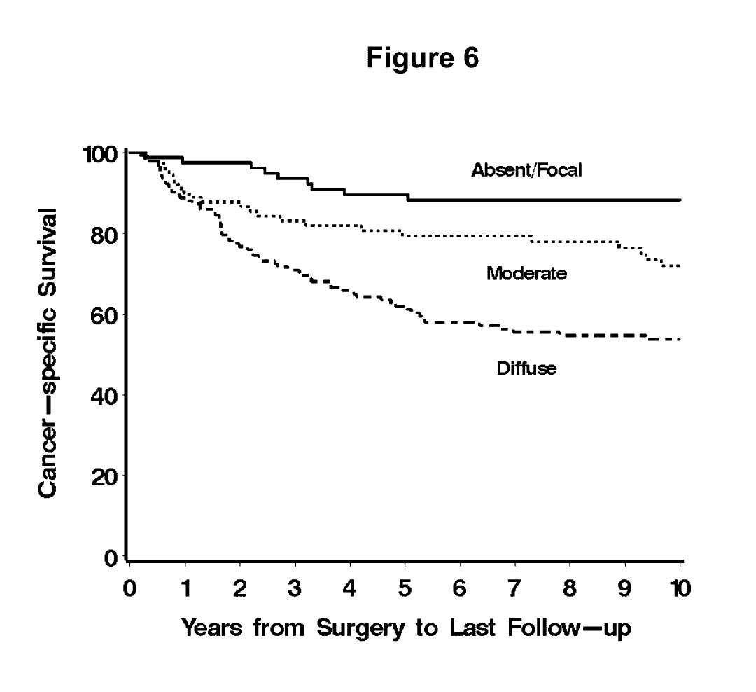

Diffuse expression in tumor vasculature was associated with adverse clinical and pathological features and increased risk of death from RCC. Univariately, the risk ratios for the associations of moderate and diffuse tumor vasculature B7-H3 expression with death from RCC (with absent/focal as the reference group) were 2.06 (95% CI 1.06-4.02; p=0.033) and 4.01 (95% CI 2.22-7.25; p<0.001), respectively. The 10-year cancer-specific survival rates for patients with absent/focal, moderate, and diffuse tumor vasculature B7-H3 expression were 88.2%, 72.0%, and 53.8% (FIG. 6). After adjusting for the SSIGN score, the risk ratios were 1.61 (95% CI 0.82-3.14; p=0.165) and 1.94 (95% CI 1.06-3.54; p=0.031), respectively. In addition, 186 tumors had some area of “normal” (i.e., non-tumor) tissue present. Of these, only 13 (7.0%) had non-tumor vasculature B7-H3 expression, and all of it was focal.

Taken together, these data demonstrate that tumor B7-H3 expression and tumor vasculature B7-H3 expression were significantly associated with death from RCC even after adjusting for each other. After adjusting for the SSIGN score, diffuse tumor vasculature B7-H3 expression was still significantly associated with death from RCC.

### Example 3—Neoadjuvant Hormonal Therapy does not Affect Expression of B7-H3 in Clinically Localized Prostate Cancer

### 1. Materials and Methods

Patient Selection—

226 patients were identified who received neoadjuvant hormonal therapy (NHT) in the form of leuprolide for a minimum of three months prior to RRP between 1990 and 1999. Patients who received other hormonal therapies or radiation therapy prior to RRP were excluded from analysis. Of these 226 cases, 61 patients who were missing clinicopathological variables were excluded from analysis. The remaining 165 men were then matched according to age at biopsy, preoperative prostate-specific antigen (PSA), clinical Gleason, clinical T-stage and year of biopsy to patients who underwent RRP during the same time period without NHT.

In addition, 50 patients were identified with a history of prostate cancer with bone metastasis from 1983-1998 who underwent bone biopsy for pathologic fractures. Of these 50 cases, 16 patients were missing slides for histopathologic review. B7-H3 expression was evaluated in samples from the remaining 34 patients. Further, expression was compared between patients who received hormone deprivation therapy prior to bone biopsy (n=23) and patients with bone metastases who did not receive hormone deprivation therapy prior to biopsy (n=11). Analyses also were conducted for a group of 9 patients who had undergone RRP for adenocarcinoma of the prostate and who subsequently had prostate biopsies of hormone refractory disease.

RRP Pathological Analysis—

Prostate glands were evaluated at the time of surgery by a standardized, limited sampling protocol using frozen section technique, followed by reevaluation the following day with hematoxylin and eosin-stained permanent sections. The surgically excised prostate was examined in the fresh state. The prostate was inked and the prostatic apex, bladder base and distal urethral margins were examined microscopically as previously described (Sebo et al. (2001) Cancer 91:2196-204). The prostate was then serially sectioned from apex to base, and at least eight standard sections through the peripheral zone of the right and left sides and one each of the right and left seminal vesicles were obtained for microscopic evaluation. The number of sections examined was contingent upon tumor volume, averaging 14 sections (range, 13 to 63). Estimates of tumor volume in cubic centimeters (cc) were calculated using the three-dimensional measurements of the tumor at the time of initial evaluation. The combination of the gross and microscopic observations served as the framework for the tumor volume estimates. Two urologic pathologists reviewed the RRP specimens, achieving consensus for extraprostatic extension, Gleason score, seminal vesicle involvement, and surgical margins. Extraprostatic extension was defined as seminal vesicle involvement or malignant cell invasion outside the prostatic capsule into adipose tissues, or tumor surrounding large nerves/ganglia beyond the prostatic capsule. These clinical and pathologic features also were combined into the GPSM score, which takes into consideration preoperative serum PSA and RRP Gleason score, seminal vesicle involvement and positive surgical margins (Blute et al. (2001) J. Urol. 165:119-125).

B7-H3 Immunohistochemistry—

Formalin-fixed, paraffin-embedded tissues were cut into 5 μm sections, deparaffinized and rehydrated in a graded series of ethanols. Antigen retrieval was performed by heating tissue sections in 1 mM EDTA (pH 8) to 121° C. using a Digital Decloaking Chamber (Biocare Medical, Walnut Creek, Calif.), cooling to 90° C., and incubating for 5 minutes. Sections were washed in Wash Buffer (Dako, Carpenteria, Calif.) before being placed onto the Autostainer Plus (Dako) to conduct the following protocol. Sections were blocked for endogenous peroxidase for 5 minutes using Endogenous Blocking Solution (Dako), washed twice, and then incubated for 5 minutes in Serum-Free Protein Block (Dako) followed by incubation for 60 minutes in purified goat anti-human B7-H3 antibody (R&D Systems, Minneapolis, Minn., 100 ug/ml) that was diluted 1:80 with DaVinci Green antibody diluent (Biocare Medical). Sections then were incubated for 15 minutes in probe from the Goat HRP-Polymer Kit (Biocare Medical #GHP516L), washed, and incubated for 15 minutes with polymer from the Goat HRP-Polymer Kit. For visualization, sections were incubated in Betazoid DAB (Biocare Medical) for 5 minutes, counterstained with hematoxylin, dehydrated in ethanol, cleared in xylene and coverslipped.

Anti-B7-H3 Competition Assay—

Goat anti-human B7-H3 antibody was combined at 1:30 with either recombinant human B7-H3-Fc fusion protein (R&D Systems), or as an additional control, P-Selectin-Fc fusion protein (BD Biosciences, San Jose, Calif.) and incubated at room temperature for 30 minutes. Immunohistochemical staining, in conditions identical to those stated above for B7-H3, was then performed with paraffin-embedded tissue sections.

Quantification of B7-H3 Expression—

The percentages of tumor and adjacent non-tumor cells that stained positive for B7-H3 were quantified in 10% increments by a urologic pathologist without knowledge of patient outcome. The intensity of B7-H3 expression was recorded as absent, weak (partial membrane staining), moderate (partial membrane and cytoplasmic staining), or marked (complete circumferential membrane and cytoplasmic staining). One-third of the specimens were independently reviewed by a second urologic pathologist to establish that the scoring of prostate tumors for B7-H3 expression was discernable and reproducible (kappa statistic 0.47).

Statistical Methods—

Clinicopathological variables were compared between NHT patients and controls using rank-sum, χ², and signed-rank test, as appropriate. Differences were considered significant when p-values were at or below 0.05. PSA progression (defined as a postoperative PSA≧0.4 ng/ml) was estimated using the Kaplan-Meier method, and compared using a log-rank test. Cox regression was used to assess the impact of tumor cell B7-H3 staining intensity on the time to PSA progression. All statistical analysis were carried out using the Statistical Analysis System software package (Cary, N.C.).

### 2. Results

Patient Demographics—

Of the original cohort of 165 matched patients, 17/165 (10.4%) of patients treated with NHT and 28/165 (17.0%) of men from the control group did not have tissue available for staining, leaving 148 patients who received NHT and 127 controls for analysis. Table 4 provides a summary of the preoperative variables used to create the matching cohort. The delay from biopsy to RRP in the patients treated with NHT (109.5 days) compared to the control group (46.6 days) was expected given the time interval during receipt of the NHT.

RRP Pathology—

Pathological outcomes from RRP for cases and controls are presented in Table 5. Interestingly, despite matching patients for preoperative variables known to predict tumor pathology, tumors from patients who received NHT were found to be significantly more likely to have a pathological Gleason score ≧7 (82/148, 55.4%), than tumors from the control group (54/127, 42.5%) (p<0.01). On the other hand, there was no difference in pathologic stage between the groups, as 98/148 (66.2%) patients treated with NHT had ≦pT2b tumors, compared with 85/127 (66.9%) patients from the control group.

Impact of NHT on B7-H3 Expression—

NHT did not significantly impact B7-H3 expression in prostate cancer specimens, as 142/148 (95.9%) tumors from patients who received NHT expressed B7-H3, compared to 122/127 (96.1%) tumors from the control group (Table 6). NHT similarly did not affect the percent of tumor cells that stained positive for B7-H3 (p=0.91) or the intensity of expression by the cancers (p=0.12; Table 6). Interestingly, NHT appeared to decrease the percent of non-tumor cells staining positive for B7-H3 (60.6% versus 68.3%, p<0.01), but did not alter the intensity of staining by the non-cancerous prostate (p=0.52)

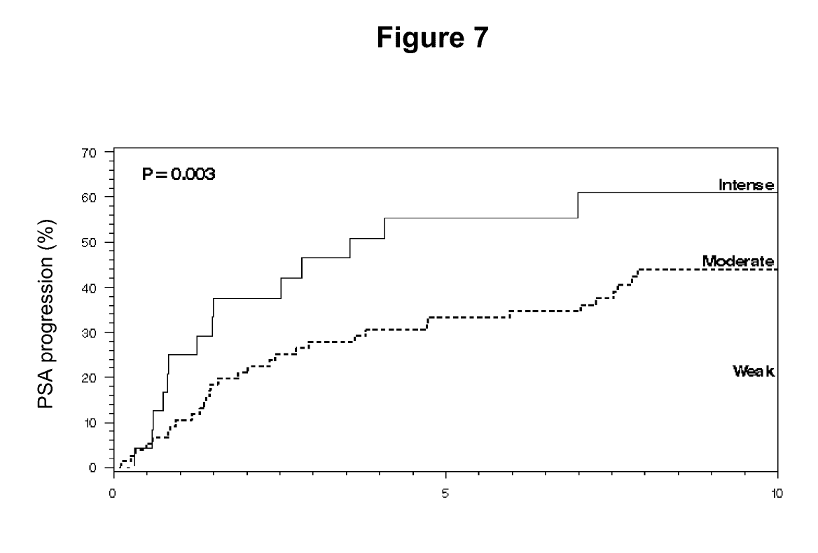

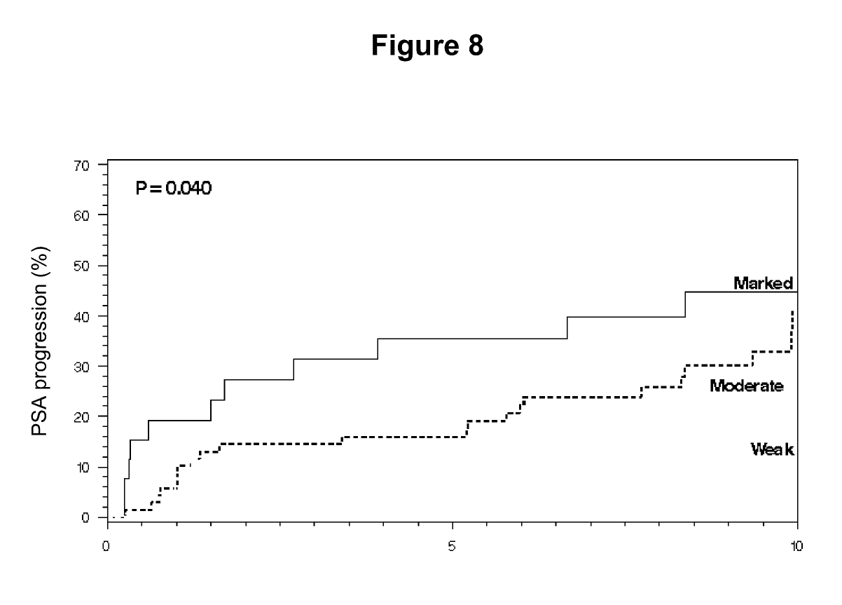

When the impact of B7-H3 expression on postoperative PSA progression was evaluated, it was found that, as demonstrated previously (Roth et al. (2007) Cancer Res. 67:7893-7900; and Zang et al. (2007) Proc. Natl. Acad. Sci. USA. 104:19458-19463), increased intensity of B7-H3 staining correlated with an increase in the 10-year PSA progression rate for both the NHT (FIG. 7) and untreated (FIG. 8) cohorts.

B7-H3 Expression in Bone Metastases—

In the cohort of 50 patients with biopsied bone metastasis, 34/50 (68%) were found to have sufficient tissue for analysis (Table 7). Within this group, 23/34 (67.6%) received hormone deprivation therapy prior to bone biopsy. Androgen deprivation therapy data was available in 37/50 patients in this cohort. The majority of these patients, 25/37 (68%) were treated with bilateral orchiectomy alone Table 8). Weak staining intensity was seen in 3/11 (27.3%) patients without hormone deprivation versus 0/23 (0%) patients with hormone deprivation. Moderate staining was noted in 3/11 (27.3%) untreated patients in the control group versus 7/23 (30.4%) treated patients, and marked intensity was noted in 5/11 (45.4%) versus 16/23 (696%) patients who were treated with hormone deprivation (p=0.04).

B7-H3 Expression in Hormone Refractory Biopsy Specimens—

Nine patients were identified in the database who initially were treated with RRP for adenocarcinoma of the prostate and who subsequently underwent biopsy for local recurrence. B7-H3 staining of tumor cells was present in 9/9 (100%) of these patients. Of this cohort, 6/9 (67%) had moderate staining, while 3/9 (33%) patients had marked staining (Table 9).

These data indicate that B7-H3 expression persists after NHT, and remains a predictor of PSA progression after RRP. These results, together with data which demonstrate continued expression in hormone refractory metastases, suggest that B7-H3 expression may be a mechanism by which select prostate cancer cells survive androgen deprivation therapy, and may represent a potential target for multimodal therapies.

TABLE 4
Preoperative demographics

| | NHT | No NHT | p |
|---|---|---|---|
| | (N = 148) | (N = 127) | value |
| Median age at | 64.6 (42.4-76.2) | 64.7 (45.1-76.9) | 0.99 |
| biopsy (range) | | | |
| Median PSA (range) | 3.1 (0.2-6.3) | 3.0 (0.3-6.5) | 0.20 |
| Biopsy Gleason score | | | 0.25 |
| ≦6 | 78 (52.7%) | 73 (57.5%) | |
| 7 | 54 (36.5%) | 45 (35.4%) | |
| 8-10 | 16 (10.8%) | 9 (7.1%) | |
| Clinical Stage | T-stage | | 0.04 |
| 1A | 0 (0%) | 1 (0.7%) | |
| 1B | 0 (0%) | 4 (2.7%) | |
| 1C | 37 (29.1%) | 42 (28.4%) | |
| 2A | 21 (16.5%) | 13 (8.8%) | |
| 2B | 29 (22.8%) | 44 (29.7%) | |
| 2C | 32 (25.2%) | 27 (18.2%) | |
| 3 | 8 (6.3%) | 17 (11.5%) | |
| Year of biopsy | | | 0.33 |
| 1990 | 1 (0.7%) | 4 (3.1%) | |
| 1991 | 1 (0.7%) | 4 (3.1%) | |
| 1992 | 3 (2%) | 4 (3.1%) | |
| 1993 | 1 (0.7%) | 1 (0.8%) | |
| 1994 | 15 (10.1%) | 8 (6.3%) | |
| 1995 | 19 (12.8%) | 19 (15%) | |
| 1996 | 28 (18.9%) | 23 (18.1%) | |
| 1997 | 25 (16.9%) | 23 (18.1%) | |
| 1998 | 36 (24.3%) | 26 (20.5%) | |
| 1999 | 19 (12.8%) | 15 (11.8%) | |
| Time from biopsy | | | <0.01 |
| to RP (days) | | | |
| Median (range) | 81.5 (14.0-791.0) | 32.0 (2.0-1149.0) | |

TABLE 5
RRP Pathology

| | NHT | No NHT | |
|---|---|---|---|
| | (N = 148) | (N = 127) | p value |
| Path Grade (GLEAS) | | | <0.01 |
| ≦6 | 62 (43.1%) | 72 (57.2%) | |
| 7 | 62 (43.1%) | 44 (34.9%) | |
| 8-10 | 20 (13.8%) | 10 (7.9%) | |
| Pathologic Stage, | | | 0.02 |
| 1997 TNM | | | |
| T2aN0 | 57 (38.5%) | 29 (22.8%) | |
| T2bN0 | 41 (27.7%) | 56 (44.1%) | |
| T3aN0 | 26 (17.6%) | 25 (19.7%) | |
| T3b4N0 | 15 (10.1%) | 8 (6.3%) | |
| TxN+ | 9 (6.1%) | 9 (7.1%) | |
| Positive surgical margin | 60 (47.2%) | 39 (26.4%) | <0.01 |
| Years RRP to death | | | 0.43 |
| or last follow-up | | | |
| Mean (SD) | 8.5 (2.21) | 8.7 (2.64) | |
| Median | 8.4 | 8.6 | |
| Q1, Q3 | 7.3, 9.9 | 7.3, 10.2 | |
| Range | (1.2-16.4) | (0.0-15.6) | |

TABLE 6
B7-H3 Expression

| | | With no | |
|---|---|---|---|
| | With | preoperative | |
| | Lupron | treatment | |
| | (N = 148) | (N = 127) | p value |
| Percent tumor cells | | | 0.91 |
| positive for B7-H3 | | | |
| Mean (min, max) | 97.5 (50, 100) | 97.1 (40, 100) | |
| Percent non-tumor cells | | | <0.01 |
| positive for B7-H3 | | | |
| Mean (min, max) | 60.6 (20, 90) | 68.3 (30, 90) | |
| With Intensity of B7-H3 | | | 0.12 |
| staining in tumor cells | | | |
| Weak | 42 (29.6%) | 26 (21.3%) | |
| Moderate | 76 (53.5%) | 70 (57.4%) | |
| Marked | 24 (16.9%) | 26 (21.3%) | |
| Intensity of B7-H3 | | | 0.52 |
| staining in non-tumor cells | | | |
| Weak | 129 (90.2%) | 107 (87.7%) | |
| Moderate | 14 (9.8%) | 15 (12.3%) | |

TABLE 7
B7-H3 Expression in Bone Metastases

| | Without AHT | With AHT | p-value |
|---|---|---|---|
| Weak | 3 (27.3%) | 0 (0.0%) | 0.043 |
| Moderate | 3 (27.3%) | 7 (30.4%) | |
| Intense | 5 (45.4%) | 16 (69.6%) | |

TABLE 8
Androgen deprivation therapy in patients with
bone metastases

| Androgen Deprivation Therapy Type | Frequency | Percent |
|---|---|---|
| Bilateral Orchiectomy | 25 | 67.57 |
| Bilateral Orchiectomy, DES | 1 | 2.70 |
| Bilateral Orchiectomy, | 1 | 2.70 |
| Flutamide (7 mos) | | |
| DES | 2 | 5.41 |
| DES, Bilateral Orchiectomy | 1 | 2.70 |
| DES, Lupron, Lupron + Flutamide | 1 | 2.70 |
| DES, Orchiectomy | 1 | 2.70 |
| Lupron, Casodex | 1 | 2.70 |
| Lupron, Flutamide | 3 | 8.11 |
| Medical | 1 | 2.70 |
| Missing | 13 | |

TABLE 9
B7-H3 Tumor Staining for
Hormone-refractory patients

| B7-H3 Tumor | | |
|---|---|---|
| Staining | Frequency | Percent |
| Moderate | 6 | 66.67 |
| Marked | 3 | 33.33 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-4. (canceled)

5. A kit for assessing the levels of B7-H3 expression in a biological sample, said kit comprising an antibody having specific binding affinity for human B7-H3.

6. The kit of claim 5, wherein said antibody is a monoclonal antibody.

7. The kit of claim 5, wherein said antibody is labeled.

8. The kit of claim 7, wherein said antibody is directly labeled.

9. The kit of claim 7, wherein said antibody is indirectly labeled.

10. The kit of claim 7, wherein said label is a radionuclide, fluorescent moiety, luminescent moiety, compound that absorbs light of a defined wavelength, or enzyme.

11. The kit of claim 10, wherein said radionuclide is selected from the group consisting of ¹²⁵I, ¹³¹I, ³⁵S, ³H, ³²P, ³³P, and ¹⁴C.

12. The kit of claim 10, wherein said fluorescent moiety is selected from the group consisting of fluorescein, fluorescein-5-isothiocyante (FITC), PerCP, rhodamine, phycoerythrin, and Texas Red.

13. The kit of claim 10, wherein said enzyme is selected from the group consisting of alkaline phosphatase and horseradish peroxidase.

14. The kit of claim 7, wherein said label is biotin.

15. The kit of claim 5, further comprising a positive control sample for detecting B7-H3.

16. The kit of claim 15, wherein said positive control sample is selected from the group consisting of a recombinant B7-H3-Fc fusion protein and a P-Selectin-Fc fusion protein.

17. A method for evaluating a biological sample from a subject with cancer, said method comprising:
detecting the presence of B7-H3 expression in a biological sample obtained from said subject using the antibody in the kit of claim 1.

18. The method of claim 17, wherein said detecting step comprises fluorescence flow cytometry or immunohistochemistry.

19. The method of claim 17, wherein said biological sample comprises renal tissue and wherein said cancer is renal cell carcinoma.

20. The method of claim 17, wherein said biological sample comprises prostate tissue and wherein said cancer is prostate cancer.

21. The method of claim 17, wherein said biological sample comprises breast tissue and wherein said cancer is breast cancer.

22. The method of claim 17, wherein said biological sample comprises ovarian tissue and wherein said cancer is ovarian cancer.

23. The method of claim 17, wherein said biological sample tissue comprises lung tissue and wherein said cancer is lung cancer.

24. The method of claim 17, wherein at least 5% of the cells in said biological sample have detectable levels of B7-H3 protein.
